(19) United States
(12) Patent Application Publication
(10) Pub. No.: US 2018/0353347 A1
(43) Pub. Date: Dec. 13, 2018

(54) WRAP AND METHOD TO PROVIDE COMPRESSION, AND PROTECTION TO BODY PART OF USER

(71) Applicant: Thomas Guy, III, Brooklyn, NY (US)

(72) Inventor: Thomas Guy, III, Brooklyn, NY (US)

(73) Assignee: Thomas Guy, III, Brooklyn, NY (US)

(21) Appl. No.: 15/615,842

(22) Filed: Jun. 7, 2017

(51) Int. Cl. A61F 13/10; A61B 5/00; A61B 5/021; A61B 5/024

(52) U.S. Cl. CPC A61F 13/108; A61F 13/107; A61F 13/064; A61B 5/02141; A61B 5/02444; A61B 5/0053

(57) ABSTRACT

Disclosed is a wrap which is removably attached with a body part of a user to provide support, compression and protection. The wrap includes first sensing unit, second sensing unit, movement unit, adjuster unit, and DC motor unit. The first sensing unit senses the physiological state of the user's body. The second sensing unit measures the dimension of the body part of the user. The body part is selected from at least one of: wrist, ankle, lower back, knee and/or combination thereof. The movement unit receives sensed information from first sensing unit, and second sensing unit to support an inflate mechanism and deflate mechanism to provide desired support and compression to the body part of the user. The adjuster unit configured with the movement unit to engage or disengage the wrap from the body part of the user. The DC motor unit which further includes a rechargeable battery powers the first sensing unit, second sensing unit, movement unit, and the adjuster unit.

### TECHNICAL FIELD

The present invention is generally related to a wrap to be worn around the wrist or ankle to provide support, compression and protection based on the sensed physiological and dimensional information of the user.

### BACKGROUND

Generally, apparatus and methods are available in the prior art which enables a user to manually tighten the exercise gloves and shoes. The tightening mechanism of the existing prior arts are completely manual and doesn't utilizes the physiological parameters of the user such as blood pressure or heartbeat to provide optimal support around the body parts under strain while doing physical activities. Further the existing apparatus and methods can tighten or compress the body of the user up to a pre-defined dimension. These apparatuses are not designed to consider and measure the dimensions and size of the user's ankle/wrist in real time to provide optimal support around the body parts under strain while doing physical activities.

Therefore, there is a need of a wrap and a method to compress and to provide optimal support around the body parts under strain while doing physical activities by utilizing the accurate physiological information and the dimensional data. Further there is also a need of a wrap and a method which can automatically inflate and deflate to to provide optimal support around the body parts under strain while doing physical activities.

Further limitations and disadvantages of conventional and traditional approaches will become apparent to one of skill in the art through comparison of described systems with some aspects of the present disclosure, as set forth in the remainder of the present application and with reference to the drawings.

### SUMMARY OF THE INVENTION

A wrap which is removably attached with at least one of a body part of a user to provide support, compression and protection is provided substantially, as shown in and/or described in connection with at least one of the figures, as set forth more completely in the claims.

The wrap includes a first sensing unit, a second sensing unit, a movement unit, an adjuster unit, and a DC motor unit. The first sensing unit senses the physiological state of the user's body. The second sensing unit measures the dimension of the body part of the user. In an embodiment, the body part is selected from at least one of: wrist, ankle, lower back, knee, and/or combination thereof. The movement unit receives the sensed information from the first sensing unit, and the second sensing unit to support an inflate mechanism and a deflate mechanism to provide desired support and compression to the body part of the user. The adjuster unit configured with the movement unit to engage or disengage the wrap from the body pan of the user. The DC motor unit which further includes a rechargeable battery powers the first sensing unit, the second sensing unit, the movement unit, and the adjuster unit.

According to embodiments illustrated herein, there is provided a method for providing support, compression and protection to at least one of a body part of a user. The method initiates with the step of sensing the physiological state of the user's body through a first sensing unit. The method further includes the step of measuring dimension of the body part of the user through a second sensing unit. In an embodiment, the body part is selected from at least one of: wrist, ankle, lower back, knee, and/or combination thereof. The method further includes the step of receiving the sensed information from the first sensing unit and the second sensing unit through a movement unit to support an inflate mechanism and a deflate mechanism to provide desired support and compression to the body part of the user. Further the method includes the step of engaging or disengaging the wrap from the body part of the user through an adjuster unit configured with the movement unit. Then the method includes the step of powering the first sensing unit, the second sensing unit, the movement unit, and the adjuster unit through a DC motor unit.

These features and advantages of the present disclosure may be appreciated by reviewing the following description of the present disclosure, along with the accompanying figures wherein like reference numerals refer to like parts.

### BRIEF DESCRIPTION OF DRAWINGS

The accompanying drawings illustrate the embodiments of systems, methods, and other aspects of the disclosure. Any person with ordinary skills in the art will appreciate that the illustrated element boundaries (e.g., boxes, groups of boxes, or other shapes) in the figures represent an example of the boundaries. In some examples, one element may be designed as multiple elements, or multiple elements may be designed as one element. In some examples, an element shown as an internal component of one element may be implemented as an external component in another, and vice versa. Furthermore, the elements may not be drawn to scale.

Various embodiments will hereinafter be described in accordance with the appended drawings, which are provided to illustrate, not limit, the scope, wherein similar designations denote similar elements, and in which:

FIG. 1 illustrates an exemplary diagram of the wrap which is removably attached with the wrist of the user to provide support, compression and protection, in accordance with at least one embodiment;

FIG. 2 illustrates an inflated view of the wrap worn around the wrist, in accordance with at least one embodiment;

FIG. 3 illustrates a deflated view of the wrap worn around the wrist, in accordance with at least one embodiment;

FIG. 4 illustrates another exemplary diagram of the wrap which is removably attached with the ankle of the user to provide support, compression and protection, in accordance with at least one embodiment;

FIG. 5 illustrates another exemplary diagram of the wrap which is removably attached with the lower back of the user to provide support, compression and protection, in accordance with at least one embodiment;

FIG. 6 illustrates another exemplary diagram of the wrap which is removably attached with the knee of the user to provide support, compression and protection, in accordance with at least one embodiment; and

FIG. 7 illustrates the flowchart of the method for providing support, compression and protection to at least one of a body part of a user, in accordance with at least one embodiment.

### DETAILED DESCRIPTION

The present disclosure is best understood with reference to the detailed figures and description set forth herein. Various embodiments have been discussed with reference to the figures. However, those skilled in the art will readily appreciate that the detailed descriptions provided herein with respect to the figures are merely for explanatory purposes, as the methods and systems may extend beyond the described embodiments. For instance, the teachings presented and the needs of a particular application may yield multiple alternative and suitable approaches to implement the functionality of any detail described herein. Therefore, any approach may extend beyond certain implementation choices in the following embodiments.

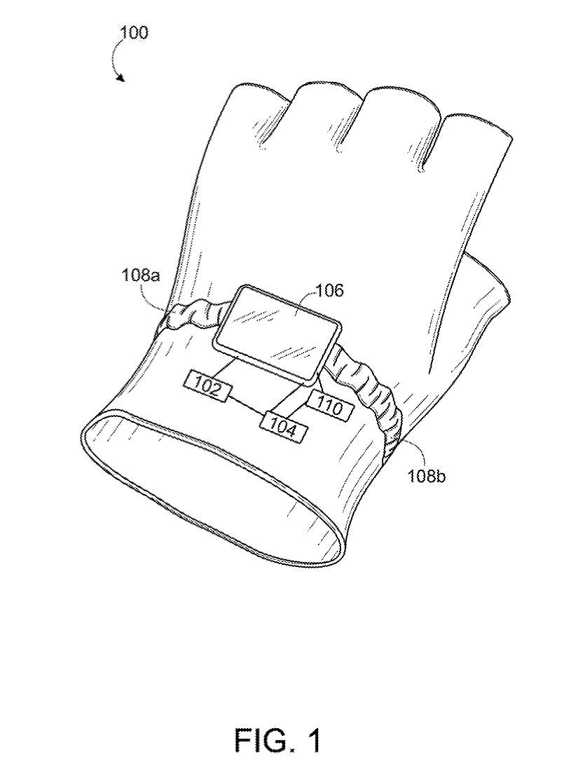

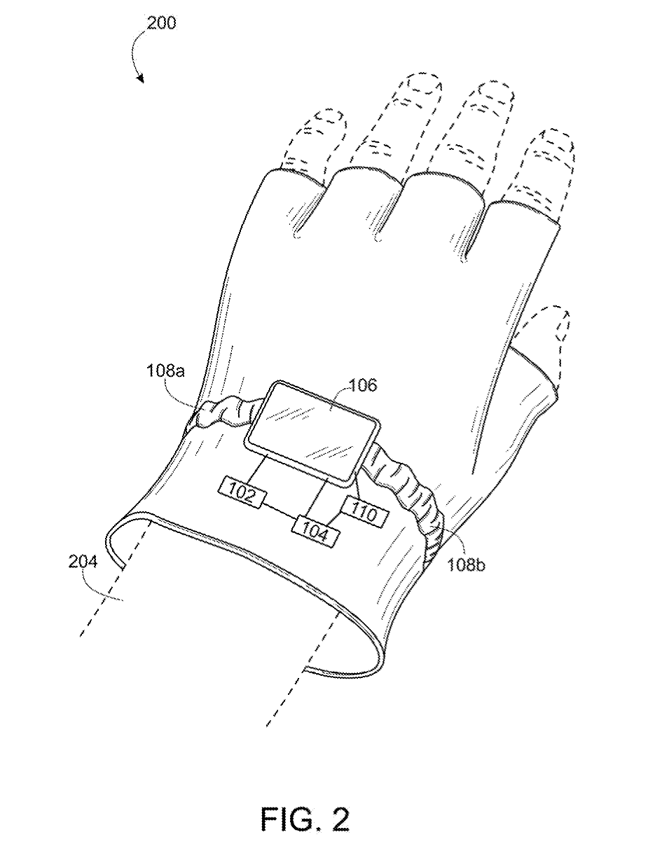

FIG. 1 illustrates an exemplary diagram 100 of the wrap which is removably attached with the wrist 204 (shown in FIG. 2) of the user to provide support, compression and protection, in accordance with at least one embodiment. The present wrap may have configured with either an exercise gloves, with an ankle shoe, lower back (preferably waist), and knee. The wrap 100 includes a first sensing unit 102, a second sensing unit 104, a movement unit 106, an adjuster unit 108a, and 108b, and a DC motor unit 110. The first sensing unit 102 senses the physiological state of the user's body. The second sensing unit 104 measures the dimension of the body part of the user. In an embodiment, the body part is selected from at least one of: wrist, ankle, lower back, knee and/or combination thereof.

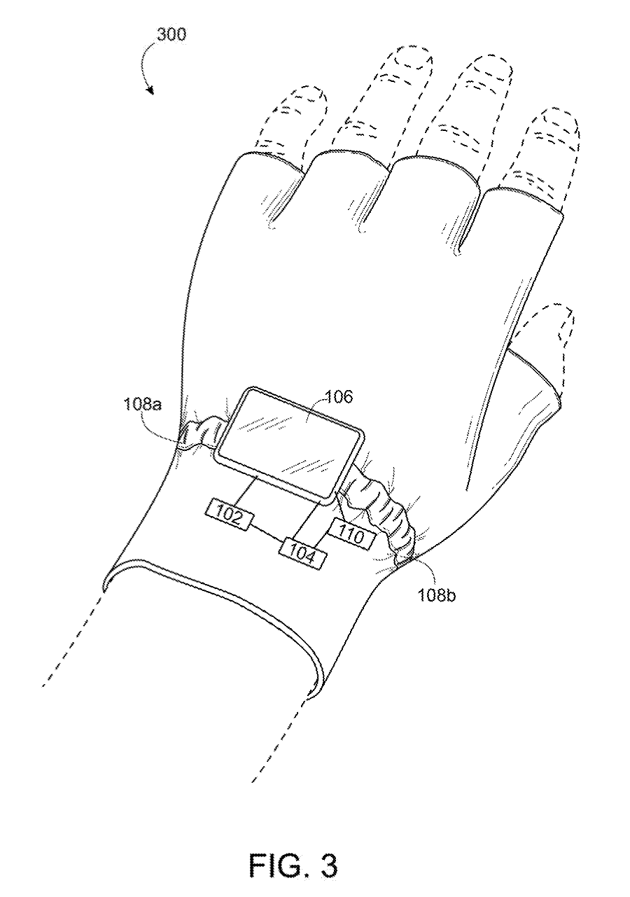

The movement unit 106 receives the sensed information from the first sensing unit 102, and the second sensing unit 104 to support an inflate mechanism (shown in FIG. 2) and a deflate mechanism (shown in FIG. 2) to provide desired support and compression to the body part of the user. FIG. 2 illustrates an inflated view 200 of the wrap worn around the wrist, in accordance with at least one embodiment. FIG. 2 is explained in conjunction with FIG. 1. The adjuster unit 108a, 108b configured with the movement unit 106 to engage or disengage the wrap from the body part of the user. FIG. 1 illustrates a deflated view 300 of the wrap worn around the wrist, in accordance with at least one embodiment. FIG. 3 is explained in conjunction with FIG. 1.

The DC motor unit 110 includes a rechargeable battery to power the first sensing unit 102, the second sensing unit 104, the movement unit 106, and the adjuster unit 108a, 108b. The movement unit 106 includes an air pump, to actuate on receiving the sensed information to provide support to the user while performing a physical activity, further the user may actuate the air pump manually. In an embodiment, the physiological state of the user's body includes blood pressure, and hear rate. The adjuster unit 108a, 108b includes at least one of: a spring, a lace, a belt, and/or combination thereof.

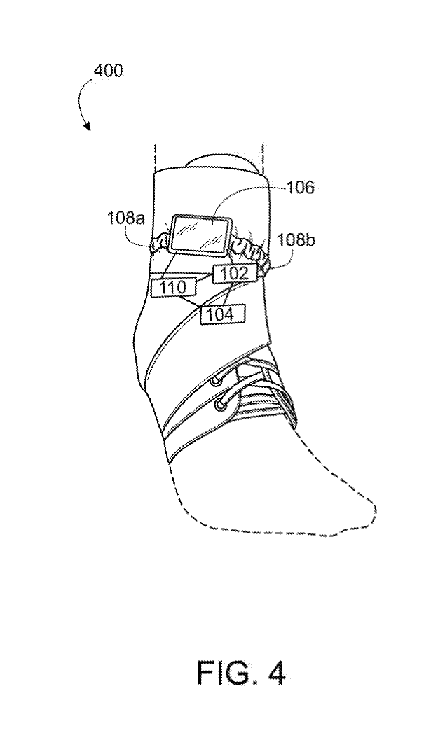

FIG. 4 illustrates another exemplary diagram 400 of the wrap which is removably attached with the ankle of the user to provide support, compression and protection, in accordance with at least one embodiment. The present wrist wraps and ankle wraps inflate to provide support, compression and protection when the user is working out or not working out. The wrap around the ankle also includes the first sensing unit 102, the second sensing unit 104, the movement unit 106, the adjuster unit 108a, and 108b, and the DC motor unit 110. The first sensing unit 102 senses the physiological state of the user's body. The second sensing unit 104 measures the dimension of the ankle of the user.

The movement unit 106 receives the sensed information from the first sensing unit 102, and the second sensing unit 104 to support an inflate mechanism and a deflate mechanism to provide desired support and compression to the ankle. The adjuster unit 108a, 108b configured with the movement unit 106 to engage or disengage the wrap from the ankle. The DC motor unit 110 powers the first sensing unit 102, the second sensing unit 104, the movement unit 106, and the adjuster unit 108a, 108b.

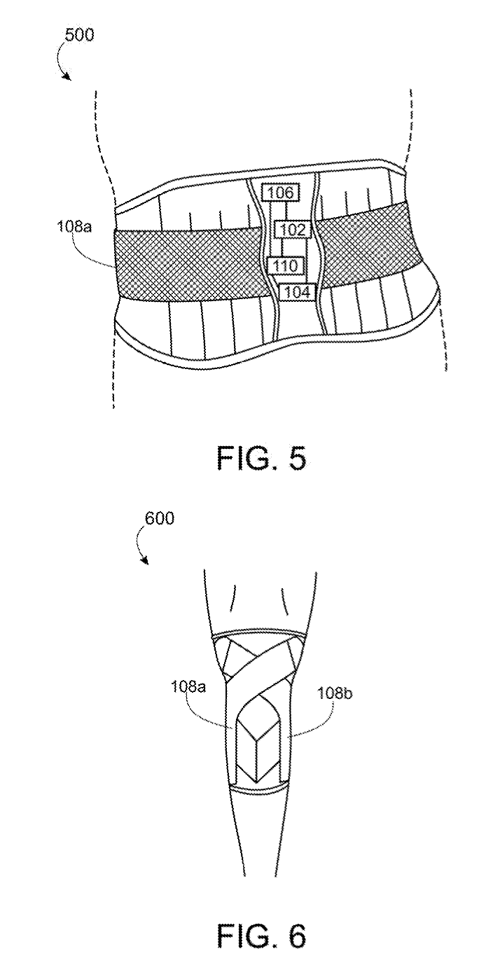

Further, FIG. 5 illustrates another exemplary diagram 500 of the wrap which is removably attached with the lower back of the user to provide support, compression and protection, in accordance with at least one embodiment. As mentioned above, the wrap utilized around the lower back also includes the first sensing unit 102, the second sensing unit 104, the movement unit 106, the adjuster unit 108a, and 108b, and the DC motor unit 110. The first sensing unit 102 senses the physiological state of the user's body. The second sensing, unit 104 measures the dimension of the lower back of the user.

The movement unit 106 receives the sensed information from the first sensing unit 102, and the second sensing unit 104 to support an inflate mechanism and a deflate mechanism to provide desired support and compression to the lower back. The adjuster unit 108a, 108b configured with the movement unit 106 to engage or disengage the wrap from the lower back. The DC motor unit 110 powers the first sensing unit 102, the second sensing unit 104, the movement unit 106, and the adjuster unit 108a, 108b. Similarly, FIG. 6 illustrates another exemplary diagram 600 of the wrap which is removably attached with the knee of the user to provide automatic support, compression and protection, in accordance with at least one embodiment.

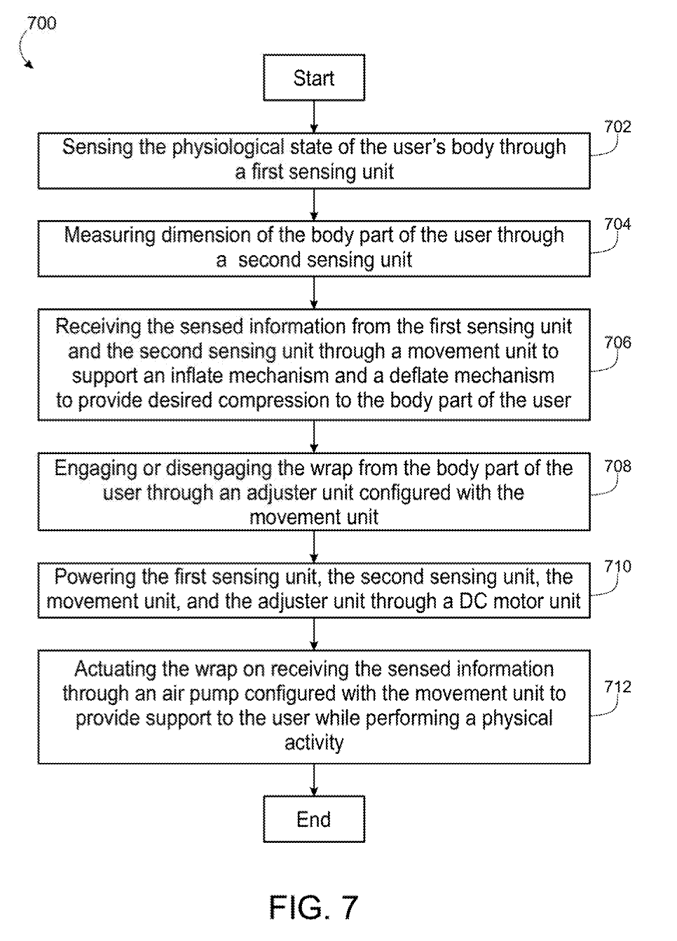

FIG. 7 illustrates the flowchart 700 of the method for providing support, compression and protection to at least one of a body part of a user, in accordance with at least one embodiment. FIG. 7 is explained in conjunction with FIG. 1, FIG. 4, FIG. 5, and FIG. 6. The method initiates with the step 702 of sensing the physiological state of the user's body through a first sensing unit. The method further includes the step 704 of measuring dimension of the body part of the user through a second sensing unit. In an embodiment, the body part is selected from at least one of wrist, ankle, lower back, knee and/or combination thereof. The method further includes the step 706 of receiving the sensed information from the first sensing unit and the second sensing unit through a movement unit to support an inflate mechanism and a deflate mechanism to provide desired support and compression to the body part of the user. Further the method includes the step 708 of engaging or disengaging the wrap from the body part of the user through an adjuster unit configured with the movement unit. Then the method includes the step 710 of powering the first sensing unit, the second sensing unit, the movement unit, and the adjuster unit through a DC motor unit.

Further the method includes the step 712 of actuating the wrap on receiving the sensed information through an air pump configured with the movement unit to provide support to the user while performing a physical activity. Additionally, the user can also actuate the air pump manually. In an embodiment, the DC motor unit includes a lithium-ion battery.

Thus, the present wrap is a part of the exercise gloves or ankle shoes and automatically inflates and deflates by utilizing an inbuilt air pump which actuates manually or automatically sensing user wrist/ankle size or blood pressure to provide support during physical activity. The present wrap acts as a grip to the ankle/wrist and solves the problem of being a non-wired, non-remote-controlled ankle or wrist compression product. The present wrap gently applies and releases the pressure without using a remote-control device or a wired assistance.

No language in the specification should be construed as indicating any non-claimed element as essential to the practice of the invention.

It will be apparent to those skilled in the art that various modifications and variations can be made to the present invention without departing from the spirit and scope of the invention. There is no intention to limit the invention to the specific form or forms enclosed. On the contrary, the intention is to cover all modifications, alternative constructions, and equivalents falling within the spirit and scope of the invention, as defined in the appended claims. Thus, it is intended that the present invention cover the modifications and variations of this invention, provided they are within the scope of the appended claims and their equivalents.

1. A wrap removably attached with at least one of a body part of a user to provide support, compression and protection, the wrap comprising:
a first sensing unit to sense the physiological state of the user's body;
a second sensing unit to measure dimension of the body part of the user, wherein the body part is selected from at least one of: wrist, ankle, lower back, knee and/or combination thereof;
a movement unit receives the sensed information from the first sensing unit and the second sensing unit to support an inflate mechanism and a deflate mechanism to provide desired support and compression to the body part of the user;
an adjuster unit configured with the movement unit to engage or disengage the wrap from the body part of the user; and
a DC motor unit to power the first sensing unit, the second sensing unit, the movement unit, and the adjuster unit.

2. The wrap according to claim 1, wherein the movement unit includes an air pump to actuate on receiving the sensed information to provide support to the user while performing a physical activity, further the user may actuate the air pump manually.

3. The wrap according to claim 1, wherein the physiological state of the user's body includes blood pressure, and heart rate.

4. The wrap according to claim 1, wherein the adjuster unit includes at least one of: a spring, a lace, a belt, and/or combination thereof.

5. The wrap according to claim 1 may configured with an exercise gloves or an ankle shoe.

6. A method for providing support, compression and protection to at least one of a body part of a user, the method comprising steps of:
sensing the physiological state of the user's body through a first sensing unit measuring dimension of the body part of the user through a second sensing unit, wherein the body part is selected from at least one of: wrist, ankle, lower back, knee and/or combination thereof;
receiving the sensed information from the first sensing unit and the second sensing unit through a movement unit to support an inflate mechanism and a deflate mechanism to provide desired support and compression to the body part of the user;
engaging or disengaging the wrap from the body part of the user through an adjuster unit configured with the movement unit; and
powering the first sensing unit, the second sensing unit, the movement unit, and the adjuster unit through a DC motor unit.

7. The method according to claim 6, further includes the step of actuating the wrap on receiving the sensed information through an air pump configured with the movement unit to provide support to the user while performing a physical activity, further the user may actuate the air pump manually.

8. The method according to claim 6, wherein the physiological state of the user's body includes blood pressure, and hear rate.

9. The method according to claim 6, wherein the adjuster unit includes at least one of a spring, a lace, a belt, and/or combination thereof.

10. The method according to claim 6, wherein the DC motor unit is a lithium-ion battery.
